(19) United States
(12) Patent Application Publication
(10) Pub. No.: US 2019/0200910 A1
(43) Pub. Date: Jul. 4, 2019

(54) TEST SYSTEM AND METHOD FOR OLFACTORY COGNITIVE ABILITY

(71) Applicant: Daegu Gyeongbuk Institute of Science and Technology, Daegu (KR)

(72) Inventors: Sang Ho LEE, Daegu (KR); WON-SEOK KANG, Daegu (KR); Che il Moon, Daegu (KR)

(73) Assignee: Daegu Gyeongbuk Institute of Science and Technology, Daegu (KR)

(21) Appl. No.: 16/226,540

(22) Filed: Dec. 19, 2018

(30) Foreign Application Priority Data
Dec. 29, 2017 (KR) 10-2017-0184212

(51) Int. Cl. A61B 5/00; A61B 5/16

(52) U.S. Cl. CPC A61B 5/4011; A61B 5/4088; A61B 5/7405; A61B 5/162; A61B 5/7275

(57) ABSTRACT

An olfactory cognitive ability test system includes an olfactory stimulator configured to accommodate an olfactory stimulating material, and an olfactory stimulation terminal configured to recognize the olfactory stimulator and to perform an olfactory cognitive ability test based on an olfactory stimulation protocol. The olfactory stimulation terminal is provided based on a mobile device, and an amount of the olfactory stimulating material or an amount of an olfactory stimulation may be adjusted to a fixed amount in the olfactory stimulator by a user's operation.

### CROSS-REFERENCE TO RELATED APPLICATION(S)

This application claims the benefit of Korean Patent Application No. 10-2017-0184212, filed on Dec. 29, 2017, in the Korean Intellectual Property Office, the disclosure of which is incorporated herein by reference.

### BACKGROUND

### 1. Field of the Invention

Example embodiments relate to an olfactory cognitive ability test system and method. More particularly, example embodiments relate to an olfactory cognitive ability test system that may include an olfactory stimulator configured to allow an olfactory stimulating material to be physically inhaled and an olfactory stimulation terminal provided based on a mobile device, to detect high-risk diseases of testees by analyzing an olfactory cognitive ability based on an olfactory stimulation protocol, and relate to an olfactory cognitive ability test method.

### 2. Description of the Related Art

Since an existing olfactory cognitive ability test system and olfactory stimulation system are significantly large in size, it is difficult to move the olfactory cognitive ability test system and olfactory stimulation system. The olfactory cognitive ability test system is relatively small in size in comparison to the olfactory stimulation system, but it is difficult to generate a smell stimulation by a fixed amount of an olfactory stimulating material at a fixed speed. Also, an amount of a stimulation continues to decrease based on a time of use of a corresponding system. In addition, errors may occur due to a poor manipulation of a cartridge or machine caused by a lack of understanding of a test system of a testee, and only a test of identifying a type of olfactory stimulating materials is possible.

Also, an olfactory cognitive ability test is clinically used to check an olfactory cognitive ability through an olfactory stick-based olfactory cognitive ability test (for example, a Korean version of the sniffin stick test (KVSS)-II, and the like), and to early diagnose geriatric depression, mild cognitive impairment (MCI), dementia, and the like, based on the examined olfactory cognitive ability. Although the olfactory cognitive ability test is excellent in terms of mobility due to use of multiple sticks, an accurate examination is limited because an olfactory stimulating material in a stick gradually disappears to provide a different level of an olfactory stimulation to each user similarly to the olfactory cognitive ability test system.

To solve the above-described issues, it is necessary to develop a simplified olfactory cognitive ability test system that may guarantee a mobility and accuracy.

For example, KR 10-1052365 B1 discloses a configuration in which an olfactory sense cartridge including an olfactory stimulus is installed in a system and in which a user directly smells a scent (odor) from the olfactory sense cartridge and inputs an estimated type of scent (odor) materials included in the olfactory sense cartridge to the system using various input devices.

### SUMMARY

Example embodiments provide an olfactory cognitive ability test system that may include an olfactory stimulator configured to allow an olfactory stimulating material to be physically inhaled and an olfactory stimulation terminal provided based on a mobile device, to detect high-risk diseases of testees by analyzing an olfactory cognitive ability based on an olfactory stimulation protocol, and provide an olfactory cognitive ability test method.

Example embodiments provide an olfactory cognitive ability test system and method that may guarantee a mobility and accuracy.

Example embodiments provide an olfactory cognitive ability test system and method that may easily change an olfactory stimulating material and that may adjust an amount of an olfactory stimulation or an amount of the olfactory stimulating material so that a testee may inhale a fixed amount of the olfactory stimulating material, to perform a more accurate olfactory stimulation.

Example embodiments provide an olfactory cognitive ability test system and method that may estimate an amount of an olfactory stimulation or an amount of an olfactory stimulating material based on a sound wave by a pressure of an olfactory stimulating material discharged from a container, to easily measure an olfactory cognitive ability at home based on a simplified configuration.

Example embodiments provide an olfactory cognitive ability test system and method that may allow a selective test to be performed based on a family history or a medical history to reduce medical expenses and to provide various types and concentrations of olfactory stimulating materials used by regions and countries.

According to an aspect, there is provided an olfactory cognitive ability test system including an olfactory stimulator configured to accommodate an olfactory stimulating material, and an olfactory stimulation terminal configured to recognize the olfactory stimulator and to perform an olfactory cognitive ability test based on an olfactory stimulation protocol, wherein the olfactory stimulation terminal is provided based on a mobile device, and wherein an amount of the olfactory stimulating material or an amount of an olfactory stimulation is adjustable to a fixed amount in the olfactory stimulator by a user's operation.

The olfactory stimulation terminal may be configured to estimate the amount of the olfactory stimulating material or the amount of the olfactory stimulation from a sound wave generated in the olfactory stimulator.

The olfactory stimulator may include a container configured to accommodate the olfactory stimulating material, a manipulator configured to expose a fixed amount of the olfactory stimulating material from the container by a user's operation, a sound wave generator configured to generate a sound wave by a pressure of the olfactory stimulating material discharged from the container, and an inhaler configured to allow the olfactory stimulating material passing through the sound wave generator to be inhaled into a nose.

The sound wave generator may include a nozzle member included in the container to spray the olfactory stimulating material at a high pressure, and a resonance member configured to allow the olfactory stimulating material discharged from the container to resonate at a standing wave. A magnitude of a sound wave generated in the nozzle member may be increased by the resonance member.

The amount of the olfactory stimulating material may be calculated in the olfactory stimulation terminal by the following equation:

Y=aX+b

In the equation, Y denotes the amount of the olfactory stimulating material, and X denotes a sound wave generation time. Also, a and b may be determined based on a size of a container that accommodates an olfactory stimulating material, a characteristic of a sound wave, or a discharge pressure of an olfactory stimulating material.

The olfactory stimulation terminal may include a sound wave detector configured to detect a sound wave generated in the olfactory stimulator. The sound wave detector may be configured to convert the sound wave to an electric signal.

The olfactory stimulation terminal may include an inputter configured to allow a testee to input a response to the olfactory stimulating material, and an olfactory cognitive ability analyzer configured to analyze an olfactory cognitive ability of the testee based on a reaction time of the testee. The reaction time of the testee may be calculated as a time interval between a time at which the sound wave is generated and a time at which the testee inputs the response.

The olfactory stimulation terminal may further include a protocol controller configured to control the olfactory stimulation protocol. The olfactory cognitive ability analyzer may be configured to analyze an olfactory cognitive ability of the testee for each function or each smell based on the olfactory stimulation protocol.

The olfactory cognitive ability analyzer may be configured to predict a disease onset possibility of the testee based on a percentage of correct answers of a testee, a reaction time of a testee, or identification information for a predetermined olfactory stimulating material.

The container may have a quick response (QR) code or a near field communication (NFC) tag to recognize a type of the olfactory stimulating material. The olfactory stimulation terminal may include an olfactory stimulating material recognizer configured to recognize the QR code or the NFC tag of the container.

According to an aspect, there is provided an olfactory cognitive ability test method including recognizing a container that accommodates an olfactory stimulating material, detecting a sound wave generated when the olfactory stimulating material is discharged from the container, calculating an amount of the olfactory stimulating material discharged from the container, receiving, as an input, a response to the olfactory stimulating material from a testee, calculating a percentage of correct answers and a reaction time of the testee from the response, and analyzing an olfactory cognitive ability of the testee based on the percentage of the correct answers, the reaction time, or identification information for a predetermined olfactory stimulating material, wherein the calculating of the amount of the olfactory stimulating material includes determining whether the amount of the olfactory stimulating material discharged from the container is a fixed amount.

The olfactory cognitive ability test method may further include, after the analyzing of the olfactory cognitive ability, analyzing a disease onset rate based on a disease database and an olfactory cognitive ability history of the testee.

The calculating of the amount of the olfactory stimulating material may include calculating the amount of the olfactory stimulating material to be directly proportional to a time during which the sound wave is generated.

The calculating of the percentage of the correct answers and the reaction time may include calculating the reaction time as a time interval between a time at which the sound wave is generated and a time at which the testee inputs the response, and determining the percentage of the correct answers based on whether the response corresponds to the olfactory stimulating material discharged from the container.

The receiving of the response may include receiving the response as an input by a touch or sound.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 illustrates a configuration of an olfactory cognitive ability test system according to an example embodiment;

FIG. 2 schematically illustrates an olfactory stimulator according to an example embodiment;

FIG. 3 illustrates an internal structure of an olfactory stimulator according to an example embodiment;

FIG. 4 illustrates a method of calculating a reaction time in an olfactory cognitive ability analyzer according to an example embodiment;

FIG. 5 is a graph showing a relationship between an amount of an olfactory stimulation and a reaction time according to an example embodiment;

FIG. 6 illustrates a process of deriving an olfactory cognitive ability or a disease onset possibility of a testee in an olfactory cognitive ability analyzer according to an example embodiment;

FIG. 7 is a flowchart illustrating an olfactory cognitive ability test method according to an example embodiment; and

FIG. 8 is a flowchart illustrating an olfactory cognitive ability test process according to an example embodiment.

### DETAILED DESCRIPTION

Hereinafter, example embodiments will be described with reference to the accompanying drawings. In the following description, the same elements will be designated by the same reference numerals although they are shown in different drawings. Also, in the following description of example embodiments, a detailed description of known functions and configurations incorporated herein will be omitted when it is deemed that such description will cause ambiguous interpretation of the example embodiments.

Also, to describe components according to example embodiments, the terms first, second, A, B, (a), (b), etc. may be used herein. These terms are merely used to distinguish one component from another, but not to define an essence, order or sequence of the components. It should be noted that if it is described in the specification that one component is “connected”, “coupled”, or “joined” to another component, it can be directly connected or coupled to the other component, or intervening components may be present.

A component included in one example embodiment and a component having a common function will be described using the same names in other example embodiments. Description of one example embodiment may be applied to other example embodiments, and overlapping detailed descriptions thereof will be omitted, unless mentioned otherwise.

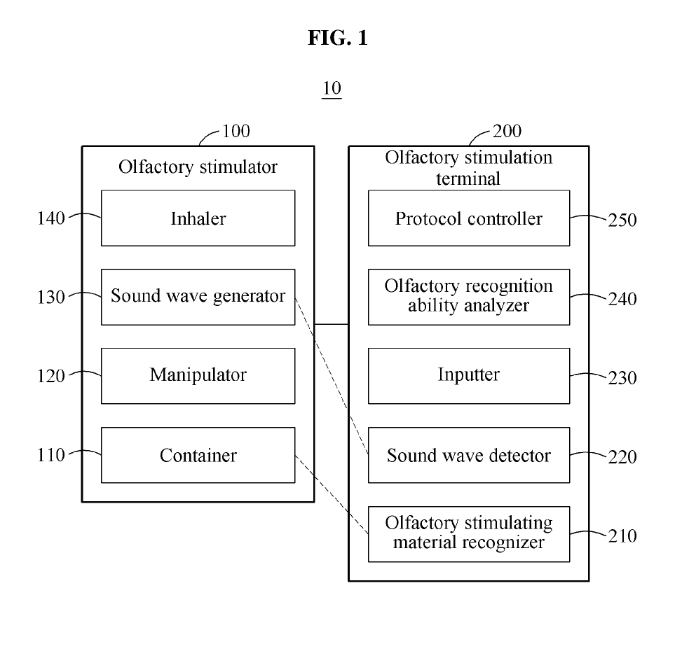

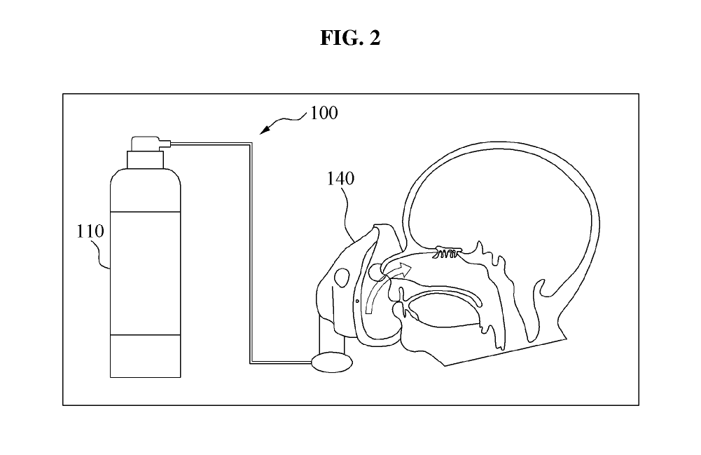

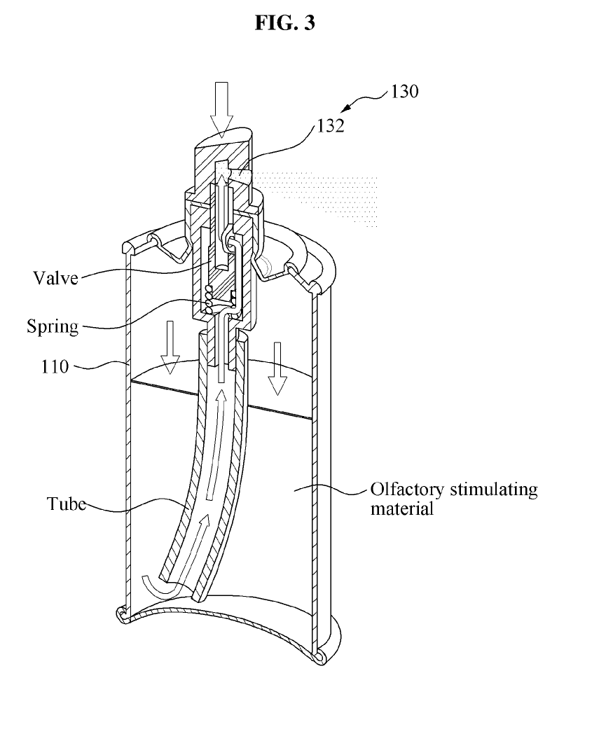

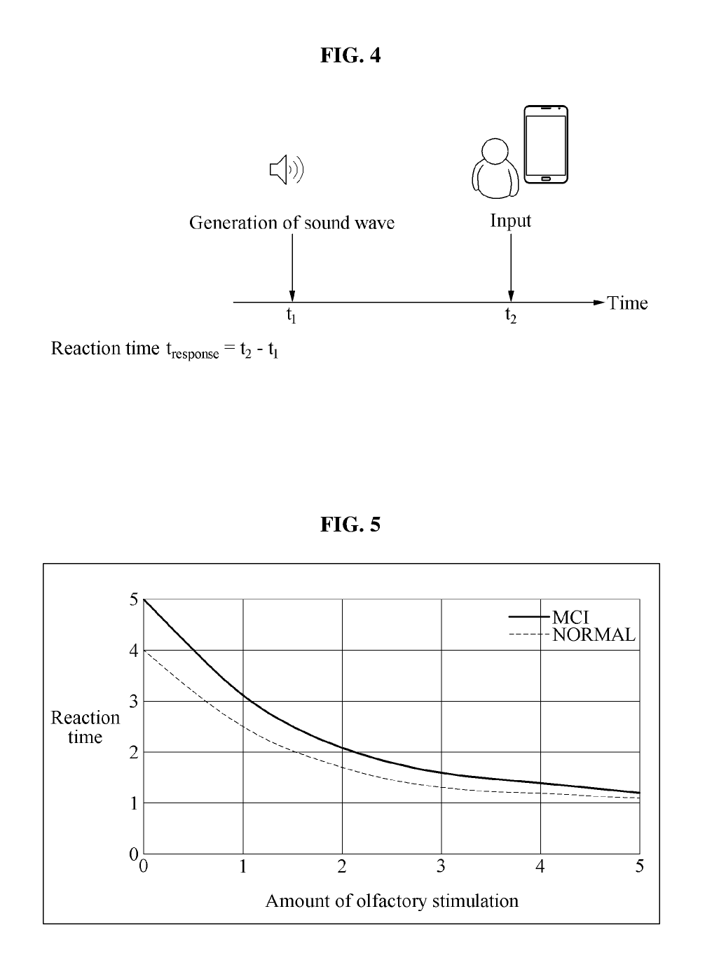

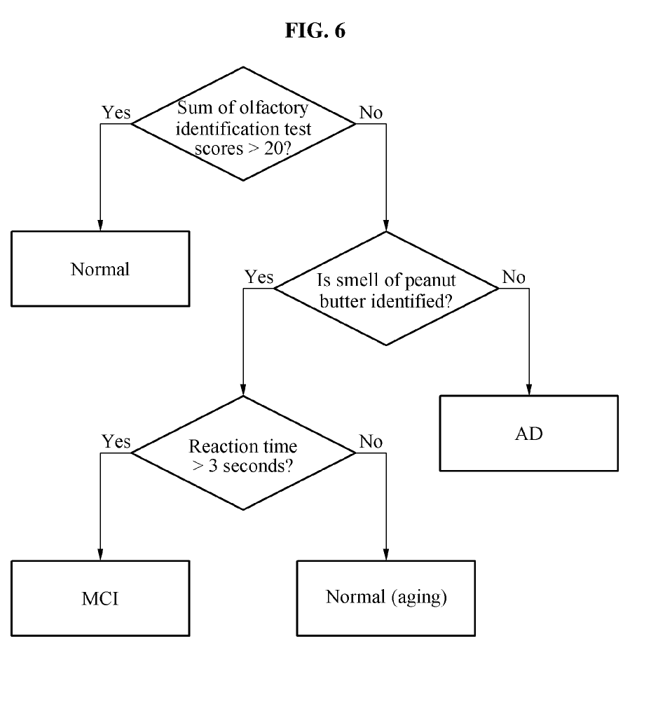

FIG. 1 illustrates a configuration of an olfactory cognitive ability test system according to an example embodiment, FIG. 2 schematically illustrates an olfactory stimulator according to an example embodiment, and FIG. 3 illustrates an internal structure of an olfactory stimulator according to an example embodiment. FIG. 4 illustrates a method of calculating a reaction time in an olfactory cognitive ability analyzer according to an example embodiment, FIG. 5 is a graph showing a relationship between an amount of an olfactory stimulation and a reaction time according to an example embodiment, and FIG. 6 illustrates a process of deriving an olfactory cognitive ability or a disease onset possibility of a testee in an olfactory cognitive ability analyzer according to an example embodiment.

Referring to FIG. 1, an olfactory cognitive ability test system 10 according to an example embodiment may include an olfactory stimulator 100 and an olfactory stimulation terminal 200.

The olfactory stimulator 100 may include a container 110, a manipulator 120, a sound wave generator 130 and an inhaler 140.

The container 110 may accommodate an olfactory stimulating material.

For example, in the container 110, the olfactory stimulating material and inhalable gas (for example, air) may be mixed and filled at a high pressure.

Also, the container 110 may have a quick response (QR) code or a near field communication (NFC) tag that may be used to recognize a type of an olfactory stimulating material and a type of gas, and accordingly the olfactory stimulation terminal 200, in particular, an olfactory stimulating material recognizer 210, may recognize the container 110.

The manipulator 120 may be included in the container 110.

The manipulator 120 may allow the olfactory stimulating material to be discharged from the container 110 to expose a fixed amount of the olfactory stimulating material to the outside.

The manipulator 120 may include, for example, a push-button manipulation member.

Thus, an amount of the olfactory stimulating material or an amount of an olfactory stimulation may be adjusted to a fixed amount by a user's operation in the olfactory stimulator 100.

Also, the container 110 may include the sound wave generator 130 configured to generate a sound wave by a pressure of an olfactory stimulating material discharged from the container 110 by the manipulator 120.

The sound wave generator 130 may allow the olfactory stimulating material discharged from the container 110 at a high pressure to resonate based on a principle of a standing wave, and may generate sound.

To this end, as shown in FIG. 3, the sound wave generator 130 may include a nozzle member 132 and a resonance member (not shown). The nozzle member 132 may be included in the container 110 to spray an olfactory stimulating material at a high pressure. The resonance member may allow the olfactory stimulating material discharged from the container 110 to resonate at a standing wave.

For example, an olfactory stimulating material may be discharged at an atmospheric pressure from a container 110 that is in a relatively low pressure state, and a sound wave may be generated due to a sound pressure difference between inside and outside the container 10 by a narrow nozzle member 132.

In this example, when a predetermined period of time elapses, a valve may be closed by a spring, and the like, and a magnitude of the sound wave generated in the nozzle member 132 may be increased by the resonance member.

Thus, an amount of an olfactory stimulating material discharged from the container 110 to air may vary. Also, the amount of the olfactory stimulating material discharged from the container 110 to air may change based on an amount of a force to press the manipulator 120 in the container 110 and a strength of a spring included in the container 110. Due to a difference between olfactory stimulating materials, an error of a stimulation test result may occur. In particular, since a difference between amounts of olfactory stimulating materials has an influence on a stimulus velocity, a stimulation by a fixed amount of olfactory stimulating materials is important.

The sound wave generated by the above-described sound wave generator 130 may be utilized to estimate or adjust an amount of an olfactory stimulating material or an amount of an olfactory stimulation, and also be utilized to measure a response time of a testee to analyze an olfactory cognitive ability of the testee.

The inhaler 140 may be connected to the container 110.

Referring to FIG. 2, the inhaler 140 may be provided to allow an olfactory stimulating material passing through the sound wave generator 130 to be inhaled into a nose. Thus, the inhaler 140 may have all structures capable of facilitating an inhalation of an olfactory stimulating material into a nose.

Referring back to FIG. 1, the olfactory stimulation terminal 200 may include an olfactory stimulating material recognizer 210, a sound wave detector 220, an inputter 230, an olfactory cognitive ability analyzer 240, and a protocol controller 250.

The olfactory stimulation terminal 200 may be provided based on a mobile device, and may include, for example, a smart terminal possessed by a testee.

The olfactory stimulating material recognizer 210 may recognize information of the QR code (image information) or the NFC tag (ID) of the container 110 as described above.

In the olfactory stimulation terminal 200, an olfactory cognitive ability may be tested based on an olfactory test protocol.

For example, during an olfactory cognitive ability test, the sound wave detector 220 may detect a sound wave generated by the olfactory stimulator 100, in particular, the sound wave generator 130.

For example, when the olfactory stimulation terminal 200 is provided as a smart terminal, the sound wave detector 220 may be a microphone included in the smart terminal, and a sound wave may be converted to an electric signal by the microphone.

In this example, the sound wave detector 220 may more accurately detect the sound wave by filtering sound waves of a specific frequency band among sound waves generated by the sound wave generator 130.

Based on the sound wave detected by the sound wave detector 220, an amount of an olfactory stimulation, or an amount of an olfactory stimulating material discharged from the olfactory stimulator 100 may be estimated.

For example, a relationship between a time during which a sound wave is generated (hereinafter, referred to as a sound wave generation time) and an amount of an olfactory stimulation may be expressed in a form of a linear function as shown below.

Y=aX+b

Y denotes the amount of the olfactory stimulating material or the amount of the olfactory stimulation. X denotes the sound wave generation time.

Also, a and b may be determined based on a size of the container 110 that accommodates an olfactory stimulating material, a pressure of the olfactory stimulating material to be discharged via the nozzle member 132, or a characteristic of a sound wave that is generated by the sound wave generator 130 or that is detected by the sound wave detector 220.

In an example, when the container 110 is relatively less used, an olfactory stimulating material may be discharged at a high pressure from the container 110, and thus a sound wave may have a large amount of high frequency components. In another example, when the container 110 is frequently used, an olfactory stimulating material may be discharged at a low pressure from the container 110, and thus a sound wave may have a large amount of low frequency components. Thus, a constant “a” may increase when a characteristic of a sound wave generated by the sound wave generator 130 or a sound wave detected by sound wave detector 220 indicates high frequency components, and may decrease when a characteristic of a sound wave generated by the sound wave generator 130 or a sound wave detected by sound wave detector 220 indicates low frequency components.

Therefore, the amount of the olfactory stimulating material discharged from the olfactory stimulator 100, in particular, the container 110 may be directly proportional to the sound wave generation time, and thus it is possible to more easily estimate the amount of the olfactory stimulating material by measuring a sound wave generation time.

When the olfactory stimulating material discharged from the olfactory stimulator 100 is perceived by a testee, the testee may input a response to the olfactory stimulating material to the inputter 230.

The response to the olfactory stimulating material may include information about whether the testee perceives the olfactory stimulating material, and the olfactory stimulating material perceived by the testee.

The inputter 230 may be provided as, for example, a button, a touch sensor or a microphone included in the olfactory stimulation terminal 200. The testee may press the button, touch the touch sensor, or use sound, to input the response to the olfactory stimulating material to the inputter 230.

For example, referring to FIG. 4, a reaction time t a testee with respect to an response of olfactory stimulating material may be calculated as a time interval between a time t₁ at which a sound wave is generated and a time t₂ at which the testee inputs a response.

The time t₁ may be a time at which a sound wave is detected first by the sound wave detector 220, and the time t₂ may be a time at which the testee inputs the response to the inputter 230.

Hereinafter, a relationship between a reaction time and an amount of an olfactory stimulation with respect to a specific olfactory stimulating material will be described with reference to FIG. 5.

Generally, when the amount of the olfactory stimulation increases, the reaction time for the olfactory stimulating material may decrease. Referring to FIG. 5, it is confirmed that patients with mild cognitive impairment (MCI) relatively slowly react to the olfactory stimulating material in comparison to a normal group. In an example in which the amount of the olfactory stimulation is “2,” when a reaction time is within 1.8 seconds, a testee may be regarded to be normal. In this example, when a reaction time exceeds 2.1 seconds, a testee may have a high possibility of being a patient with MCI due to an abnormality in an olfactory cognitive function.

The olfactory cognitive ability analyzer 240 may analyze an olfactory cognitive ability of a testee using an olfactory stimulation protocol, based on a reaction time of the testee, as described above.

The olfactory cognitive ability analyzer 240 may check olfactory cognitive abilities of testees for each function and each smell based on the olfactory stimulation protocol, and may analyze a comprehensive olfactory cognitive ability based on the olfactory cognitive abilities.

Also, the olfactory cognitive ability analyzer 240 may predict a possibility of an onset of a specific disease based on whether an olfactory capability of a testee is normal, and a change in the olfactory capability for each function and for each olfactory stimulating material. For example, the olfactory cognitive ability analyzer 240 may predict a disease onset possibility of a testee based on a percentage of correct answers of the testee, a reaction time of the testee, or identification information for a specific olfactory stimulating material.

For example, referring to FIG. 6, the olfactory cognitive ability analyzer 240 may form a decision tree based on a percentage of correct answers and a reaction time of a testee, or identification information for a specific olfactory stimulating material, and may analyze an olfactory cognitive ability.

When a sum of olfactory identification test scores as a percentage of correct answers of a testee is equal to or greater than “20,” the testee may be determined to be normal. When the sum of the olfactory identification test scores is less than “20,” a next operation may be performed.

When the sum of the olfactory identification test scores is less than “20” and when an identification of a smell of peanut butter by a left nose fails, a possibility of an onset of Alzheimer's disease (AD) of the testee may be predicted. When the smell of the peanut butter is identified by the left nose, a next operation may be performed.

When the smell of the peanut butter is identified by the left nose and when a reaction time exceeds 3 seconds, the testee may be predicted to have MCI. When the reaction time is less than or equal to 3 seconds, the testee may be predicted to be normal (aging).

The olfactory stimulation protocol used to analyze an olfactory cognitive ability in the olfactory cognitive ability analyzer 240 may be controlled by the protocol controller 250.

The protocol controller 250 may form various decision trees, for example, the decision tree of FIG. 6, so that an olfactory cognitive ability of a testee may be analyzed for each function or each smell based on the olfactory stimulation protocol.

Thus, based on a scheme of configuring the olfactory stimulation protocol in the protocol controller 250, it is possible to variously predict a possibility of an onset of a specific disease as well as whether an olfactory cognitive ability is normal in the olfactory cognitive ability analyzer 240.

Therefore, the olfactory cognitive ability test system configured as described above may be used to easily change an olfactory stimulating material and to adjust an amount of an olfactory stimulation, to enable a more accurate olfactory stimulation. Also, an olfactory stimulator may be miniaturized and an olfactory stimulation terminal may be configured based on a mobile device, and thus it is possible to easily measure an olfactory cognitive ability at home, and possible to reduce medical expenses by performing a selective test based on a family history or medical history. Also, it is possible to provide various types and concentrations of olfactory stimulating materials used by regions and countries.

The olfactory cognitive ability test system has been described above. Hereinafter an olfactory cognitive ability test method according to an example embodiment will be described.

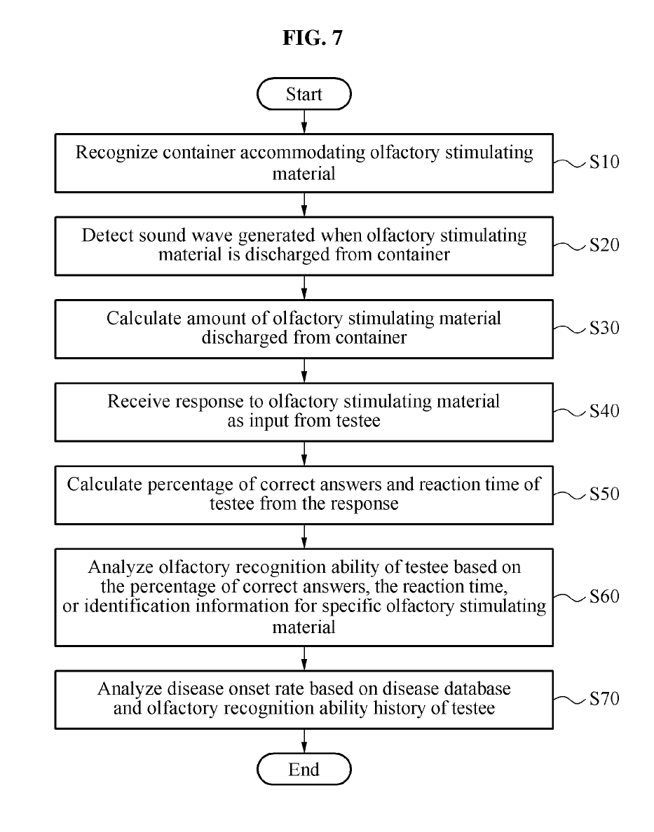

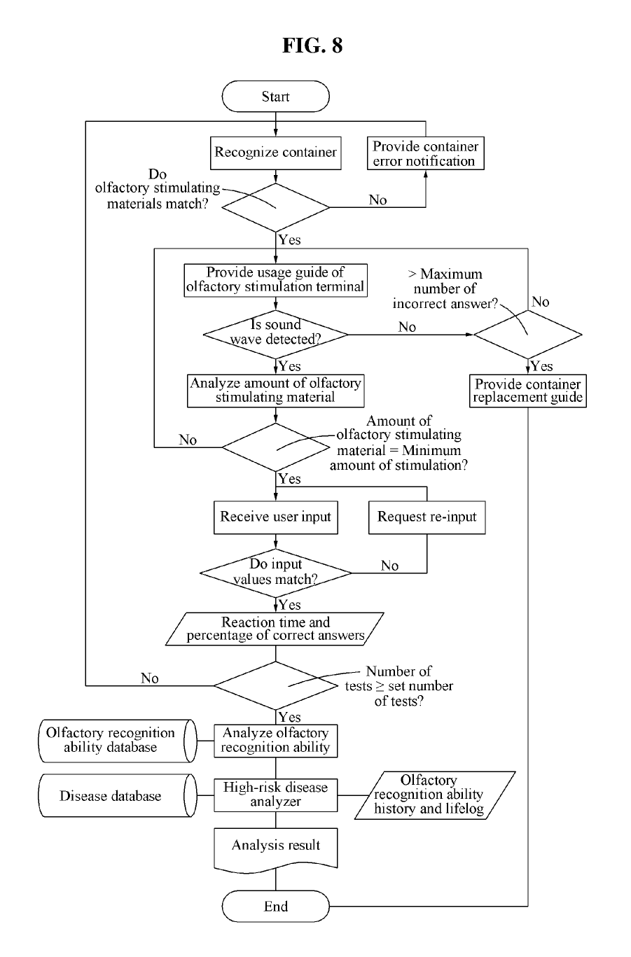

FIG. 7 is a flowchart illustrating an olfactory cognitive ability test method according to an example embodiment, and FIG. 8 is a flowchart illustrating an olfactory cognitive ability test process according to an example embodiment.

The olfactory cognitive ability test method may be performed as shown in FIGS. 7 and 8 below.

Referring to FIG. 7, in operation S10, a container that accommodates an olfactory stimulating material is recognized.

Since the container has a QR code or an NFC tag, an olfactory stimulating material recognizer of an olfactory stimulation terminal may recognize the container.

The QR code or the NFC tag may include information about a specific olfactory stimulating material, and accordingly the olfactory stimulating material recognizer may recognize a type of olfactory stimulating materials by recognizing the QR code or the NFC tag.

For example, when a container that accommodates an olfactory stimulating material is recognized and when the olfactory stimulating material in the container matches an olfactory stimulating material that is based on an olfactory stimulation protocol, a usage guide of the olfactory stimulation terminal may be provided. When the olfactory stimulating materials do not match, a container error notification may be provided to a testee.

In operation S20, a sound wave generated when the olfactory stimulating material is discharged from the container is detected.

For example, a sound wave may be generated when the olfactory stimulating material is discharged from the container, and may be detected by a sound wave detector, for example, a microphone, included in the olfactory stimulation terminal.

In operation S30, an amount of the olfactory stimulating material discharged from the container is calculated.

For example, the amount of the olfactory stimulating material discharged from the container may be directly proportional to a sound wave generation time. The amount of the olfactory stimulating material may be calculated based on a sound wave generation time, a size of a container that accommodates an olfactory stimulating material, a discharge pressure of the olfactory stimulating material, or a characteristic of a sound wave.

In this example, when the amount of the olfactory stimulating material discharged from the container is calculated, whether the amount of the olfactory stimulating material discharged from the container is a fixed amount may be determined. When the amount of the olfactory stimulating material discharged from the container is the fixed amount, an olfactory cognitive ability may be analyzed based on the olfactory stimulation protocol in the olfactory stimulation terminal. When the amount of the olfactory stimulating material discharged from the container is not the fixed amount or not a minimum amount of a stimulation, the olfactory stimulating material may be discharged from the container again.

In operation S40, a response to the olfactory stimulating material is received as an input from the testee.

For example, the response may be received by a touch or sound using a touch sensor or a microphone included in the olfactory stimulation terminal.

For example, the response may include information about whether the testee perceives the olfactory stimulating material, and the olfactory stimulating material perceived by the testee.

In operation S50, a percentage of correct answers and a reaction time of the testee are calculated from the response.

The reaction time of the testee may be calculated as a time interval between a time at which a sound wave is generated and a time at which the testee inputs the response. The percentage of correct answers may be determined based on whether the response corresponds to the olfactory stimulating material discharged from the container.

In operation S60, an olfactory cognitive ability of the testee is analyzed based on the reaction time, the percentage of correct answers or identification information for a specific olfactory stimulating material. In operation S70, a disease onset rate is analyzed based on a disease database and an olfactory cognitive ability history of the testee.

The olfactory cognitive ability of the testee may be analyzed based on an olfactory cognitive ability database. The olfactory cognitive ability database may include standard data on olfactory cognitive abilities by age, gender and region.

For example, the olfactory cognitive ability of the testee may be analyzed through a comparison between the data included in the olfactory cognitive ability database and the reaction time, the percentage of correct answers or the identification information for the specific olfactory stimulating material.

Also, the disease onset rate may be analyzed based on the disease database and the olfactory cognitive ability history of the testee. A lifelog and the olfactory cognitive ability history may be used to analyze a disease with a high incidence based on environmental factors (for example, an industrial area or a residential area) and physical factors (for example, activity information or sleep information) of the testee, during an analysis of high-risk diseases.

The disease database may also include data on a disease that is frequently occur due to a loss of an olfactory function for a specific smell, and may be used to identify diseases in a high risk group.

For example, the disease database is shown in Table 1 below.

TABLE 1

| | Olfactory stimulating | | |
|---|---|---|---|
| | material | Gender | Age |
| Disease A | Peanut butter | Female | 60s/70s |
| (AD) | | | |
| Disease B | Lemon flavor/cheese | Male/Female | 50s |
| (MCI) | flavor | | |

Using the olfactory cognitive ability test method as described above, it is possible to guarantee a mobility and accuracy, possible to easily change an olfactory stimulating material and possible to adjust an amount of an olfactory stimulation or an amount of the olfactory stimulating material so that a testee may inhale a fixed amount of the olfactory stimulating material, thereby performing a more accurate olfactory stimulation.

According to example embodiments, using an olfactory cognitive ability test system and method, an olfactory stimulator configured to allow an olfactory stimulating material to be physically inhaled and an olfactory stimulation terminal provided based on a mobile device may be included, and thus it is possible to detect high-risk diseases of testees by analyzing an olfactory cognitive ability based on an olfactory stimulation protocol.

According to example embodiments, using an olfactory cognitive ability test system and method, it is possible to guarantee a mobility and accuracy.

According to example embodiments, using an olfactory cognitive ability test system and method, an olfactory stimulating material may be easily changed and an amount of an olfactory stimulation or an amount of the olfactory stimulating material may be adjusted so that a testee may inhale a fixed amount of the olfactory stimulating material, and thus it is possible to perform a more accurate olfactory stimulation.

According to example embodiments, using an olfactory cognitive ability test system and method, an amount of an olfactory stimulation or an amount of an olfactory stimulating material may be estimated based on a sound wave by a pressure of an olfactory stimulating material discharged from a container, and a configuration may be simplified, and thus it is possible to easily measure an olfactory cognitive ability at home.

According to example embodiments, using an olfactory cognitive ability test system and method, a selective test may be performed based on a family history or a medical history, and thus it is possible to reduce medical expenses and to provide various types and concentrations of olfactory stimulating materials used by regions and countries.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An olfactory cognitive ability test system comprising:
an olfactory stimulator configured to accommodate an olfactory stimulating material; and
an olfactory stimulation terminal configured to recognize the olfactory stimulator and to perform an olfactory cognitive ability test based on an olfactory stimulation protocol,
wherein the olfactory stimulation terminal is provided based on a mobile device, and
wherein an amount of the olfactory stimulating material or an amount of an olfactory stimulation is adjustable to a fixed amount in the olfactory stimulator by a user's operation.

2. The olfactory cognitive ability test system of claim 1, wherein the olfactory stimulation terminal is configured to estimate the amount of the olfactory stimulating material or the amount of the olfactory stimulation from a sound wave generated in the olfactory stimulator.

3. The olfactory cognitive ability test system of claim 1, wherein the olfactory stimulator comprises:
a container configured to accommodate the olfactory stimulating material;
a manipulator configured to expose a fixed amount of the olfactory stimulating material from the container by a user's operation;
a sound wave generator configured to generate a sound wave by a pressure of the olfactory stimulating material discharged from the container; and
an inhaler configured to allow the olfactory stimulating material passing through the sound wave generator to be inhaled into a nose.

4. The olfactory cognitive ability test system of claim 3, wherein
the sound wave generator comprises:
a nozzle member included in the container to spray the olfactory stimulating material at a high pressure; and
a resonance member configured to allow the olfactory stimulating material discharged from the container to resonate at a standing wave, and
a magnitude of a sound wave generated in the nozzle member is increased by the resonance member.

5. The olfactory cognitive ability test system of claim 1, wherein the amount of the olfactory stimulating material is calculated in the olfactory stimulation terminal by the following equation:
Y=aX+b,
in which Y denotes the amount of the olfactory stimulating material, X denotes a sound wave generation time, and a and b are determined based on a size of a container that accommodates an olfactory stimulating material, a characteristic of a sound wave, or a discharge pressure of an olfactory stimulating material.

6. The olfactory cognitive ability test system of claim 2, wherein
the olfactory stimulation terminal comprises a sound wave detector configured to detect a sound wave generated in the olfactory stimulator, and
the sound wave detector is configured to convert the sound wave to an electric signal.

7. The olfactory cognitive ability test system of claim 2, wherein the olfactory stimulation terminal comprises:
an inputter configured to allow a testee to input a response to the olfactory stimulating material; and
an olfactory cognitive ability analyzer configured to analyze an olfactory cognitive ability of the testee based on a reaction time of the testee,
wherein the reaction time of the testee is calculated as a time interval between a time at which the sound wave is generated and a time at which the testee inputs the response.

8. The olfactory cognitive ability test system of claim 7, wherein
the olfactory stimulation terminal further comprises a protocol controller configured to control the olfactory stimulation protocol, and
the olfactory cognitive ability analyzer is configured to analyze an olfactory cognitive ability of the testee for each function or each smell based on the olfactory stimulation protocol.

9. The olfactory cognitive ability test system of claim 7, wherein the olfactory cognitive ability analyzer is configured to predict a disease onset possibility of the testee based on a percentage of correct answers of a testee, a reaction time of a testee, or identification information for a predetermined olfactory stimulating material.

10. The olfactory cognitive ability test system of claim 3, wherein
the container has a quick response (QR) code or a near field communication (NFC) tag to recognize a type of the olfactory stimulating material, and
the olfactory stimulation terminal comprises an olfactory stimulating material recognizer configured to recognize the QR code or the NFC tag of the container.

11. An olfactory cognitive ability test method comprising:
recognizing a container that accommodates an olfactory stimulating material;
detecting a sound wave generated when the olfactory stimulating material is discharged from the container;
calculating an amount of the olfactory stimulating material discharged from the container;
receiving a response to the olfactory stimulating material as an input from a testee;
calculating a percentage of correct answers and a reaction time of the testee from the response; and
analyzing an olfactory cognitive ability of the testee based on the percentage of the correct answers, the reaction time, or identification information for a predetermined olfactory stimulating material,
wherein the calculating of the amount of the olfactory stimulating material comprises determining whether the amount of the olfactory stimulating material discharged from the container is a fixed amount.

12. The olfactory cognitive ability test method of claim 11, further comprising, after the analyzing of the olfactory cognitive ability:
analyzing a disease onset rate based on a disease database and an olfactory cognitive ability history of the testee.

13. The olfactory cognitive ability test method of claim 11, wherein the calculating of the amount of the olfactory stimulating material comprises calculating the amount of the olfactory stimulating material to be directly proportional to a time during which the sound wave is generated.

14. The olfactory cognitive ability test method of claim 11, wherein the calculating of the percentage of the correct answers and the reaction time comprises:
calculating the reaction time as a time interval between a time at which the sound wave is generated and a time at which the testee inputs the response; and
determining the percentage of the correct answers based on whether the response corresponds to the olfactory stimulating material discharged from the container.

15. The olfactory cognitive ability test method of claim 11, wherein the receiving of the response comprises receiving the response as an input by a touch or sound.
